(19) United States
(12) Patent Application Publication
(10) Pub. No.: US 2007/0111980 A1
(43) Pub. Date: May 17, 2007

(54) Process for preparing pure cephalosporine intermediates

(76) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Nagabelli Murali, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/565,086

(22) PCT Filed: Jul. 16, 2004
(86) PCT No.: PCT/IN04/00209

§ 371 Date: Jan. 17, 2006

(51) Int. Cl. C07D 501/14; A61K 31/545

(52) U.S. Cl. 514/202; 540/222

(57) ABSTRACT

The present invention relates to a process for preparing key intermediates for cephalosporin antibiotics substantially free of undesired Δ² isomer. Thus, 7-aminocephalosporanic acid (7-ACA) is silylated with hexamethyldisilazane in cyclohexane at reflux temperature. (6R,7R)-3-[(Acetyloxy)methyl]-7-(trimethylsilyl)aminoceph-3-em-4-oic acid obtained is reacted with the mixture of N-methylpyrrolidine and trimethylsilyl iodide in cyclohexane, desilylated with isopropyl alcohol and treated with hydrochloric acid to obtain [6R-(6α,7β)]-1-[[7-Amino-2-carboxy-8-oxo-5-thia-1-azabicyclo[4.2.0]oct-2-en-3-yl]methyl]-1-methylpyrrolidinium inner salt hydrochloride. [6R-(6α,7β)]-1-[[7-Amino-2-carboxy-8-oxo-5-thia-1-azabicyclo[4.2.0]oct-2-en-3-yl]methyl]-1-methylpyrrolidinium inner salt hydrochloride is N-acylated with syn-2-(2-aminothiazol-4-yl)-2-methoxyimino acetic acid 2-benzothiazolyl thioester (MAEM) followed by treatment with hydrochloric acid to give cefepime dihydrochloride monohydrate.

### FIELD OF THE INVENTION

The present invention relates to a process for preparing key intermediates for cephalosporin antibiotics substantially free d undesired Δ² isomer. According to the novel process, no chromatographic separations are required for isolating Δ² isomer thereby increasing the productivity. Moreover the novel process avoids the use of expensive, environmentally hazardous fluorochlorocarbons such as freon. Thus, the novel process is environmentally safe, less expensive and commercially viable.

### BACKGROUND OF THE INVENTION

U.S. Pat. No. 4,868,294 described crystalline temperature stable hydrochloride or hydroiodide salt of a compound of formulas: 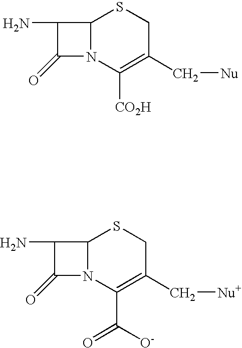
which is substantially free of the corresponding Δ² isomer, wherein
Nu is 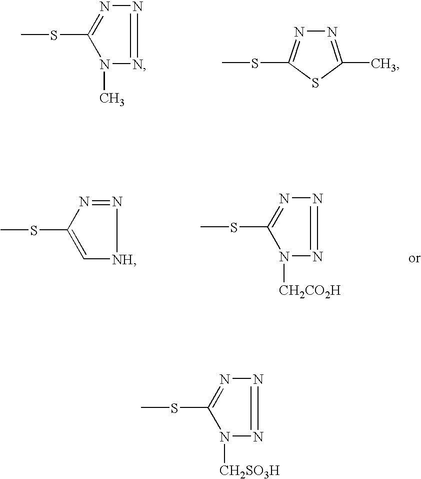
and

Nu⁺ is

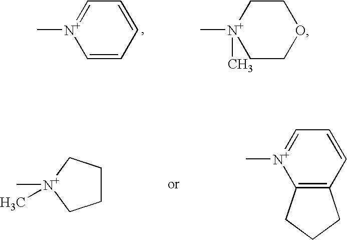

These compounds are key intermediates for the conversion by acylation into broad spectrum cephalosporin antibiotics which are substantially free of Δ² isomer.

Various cephalosporin antibiotics were disclosed in many patents, some of which are U.S. Pat. No. 4,406,899, U.S. Pat. No. 4,168,309, U.S. Pat. No. 4,223,135, U.S. Pat. No. 4,336,253 and U.S. Pat. No. 4,379,787.

J. Organic Chemistry 1988, 53, 983-991 described the effect of halogenated solvents, acetonitrile and toluene on the formation of Δ² Isomer during the preparation of the key intermediates such as those mentioned above.

U.S. Pat. No. 4,910,301 disclosed temperature stable crystalline salts of 7-[α-(2-aminothiazol-4-yl)-α-(Z)-methoxyiminoacetamidol-3-[(1-methyl-1-pyrrolidinio)methyl]-3-cephem-4-carboxylate (cefepime). These salts Include among others cefepime dihydrochloride monohydrate and cefepime sulfuric acid salt.

According to U.S. Pat. No. 4,868,294, the key intermediates substantially free of Δ² Isomer mentioned above can be prepared by carrying out the reactions according to the following reaction scheme in freon (1,1,2-trichlorotrifluoroethane) solvent medium:

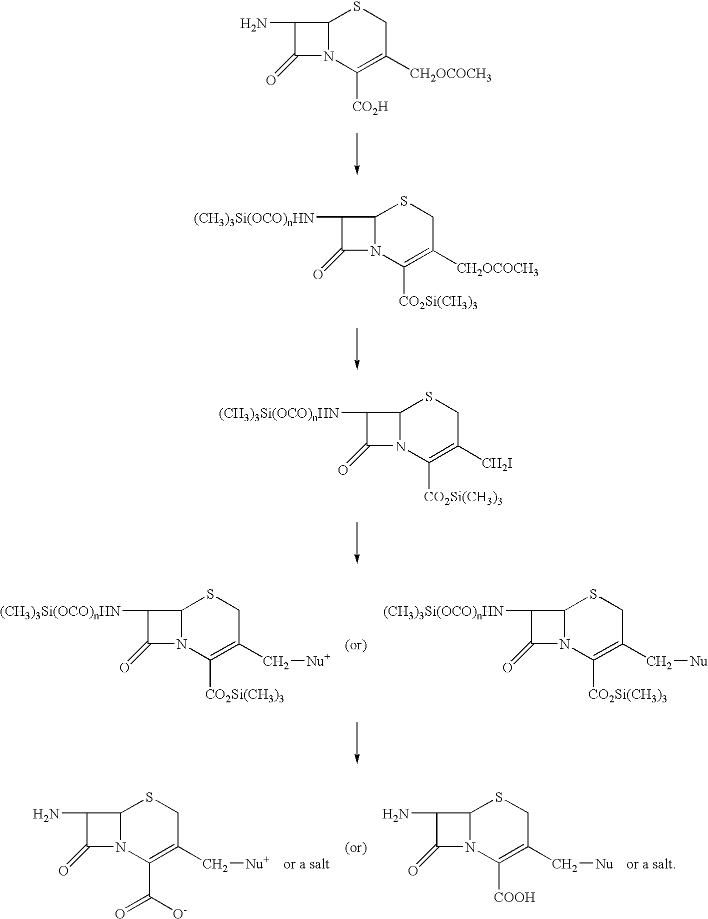

It is known that freon is environmentally hazardous chlorofluoro carbon and is expensive.

U.S. Pat. No. 5,441,874 and EP patent No. 0162395 described processes for preparing some cephalosporin antibiotics.

U.S. Pat. No. 5,594,130 described preparation d cefepime. using syn-isomer of 2-2-aminothiazol-4-yl)-2-methoxyimino acetyl chloride hydrochloride.

U.S. Pat. No. 4,680,389 described stable crystalline di (1-methyl-2-pyrrolidinone) and di (Nrformyl pyrrolidine) adducts of cephalosporin derivatives such as cefepime.

We have found that the formation of undesired Δ² isomer in the preparation of key intermediates for cephalosporin antibiotics can be reduced or avoided with the use of cyclohexane as solvent. According to the novel process, no chromatographic separations are required for isolating Δ² isomer thereby increasing the productivity. Moreover the novel process avoids the use of expensive, environmentally hazardous fluorochlorocarbons such as freon. Thus, the novel process is environmentally safe, less expensive and commercially viable.

### DETAILED DESCRIPTION OF THE INVENTION

According to the present invention, a process is provided for preparing the compounds of formulas—I(i) & I(ii) substantially free of the corresponding Δ² Isomer; or salts thereof. 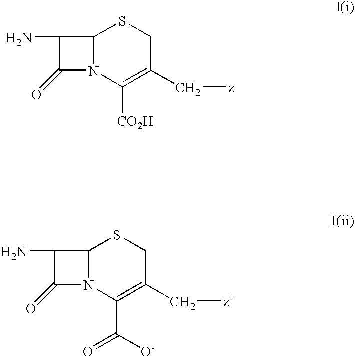
wherein
Z is 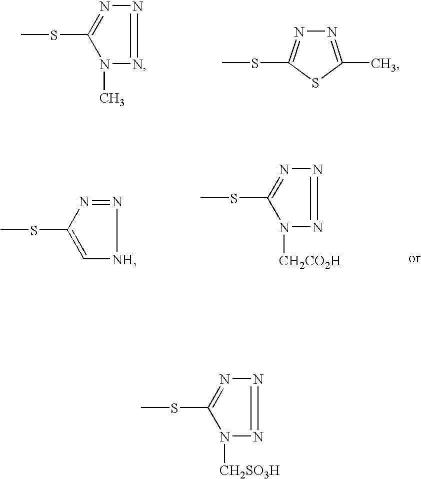
and Z⁺ is

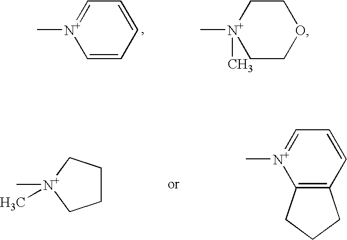

The preferred compound prepared according to the present invention is the compound of formula I(ii), wherein
Z⁺ is

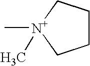

and is represented by the formula II: 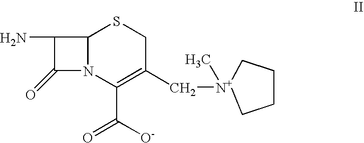
and a salt thereof.

Preferable salts are hydrochloride and hydroiodide salts.

The compound of formula II may be prepared by treating a solution of the compound of the formula III: 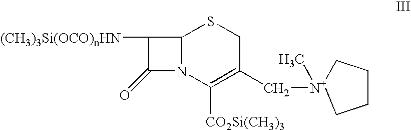
wherein n=0 or 1,
in cyclohexane with a C₁-C₄-alkanol or water to remove silyl protecting groups. The compounds of formula II are preferably converted into a salt. The compound of formula III, wherein n=0 is the preferred compound and is represented by formula IHa:

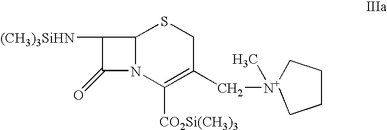

The reaction is carried out at a temperature of from about −10° C. to about 45° C., preferably at a temperature of from about CPC to about 25° C., and more preferably at a temperature of from about 0° C. to about 10° C. Preferable alcohols are isopropyl alcohol, methanol and ethanol, more preferable being isopropyl alcohol. From about 1 to about 5 equivalents of c-i-C₄-alkanol are used per equivalent of compound III.

The compounds of the formula III may be prepared by reacting a solution of the compounds of the formula IV: 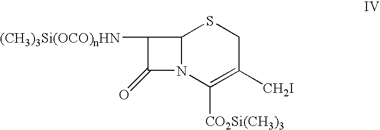
wherein n =0 or 1,
in a cydohexane with N-methyl pyrrolidine. The compound of formula IV, wherein n=0 is the preferred compound and is represented by formula IVa:

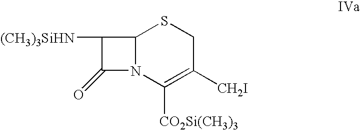

It has been surprisingly found that when cyclohexane is used as solvent, compound III obtained is substantially free of the Δ² isomer. It is known from U.S. Pat. No. 4,868,294 that when the solvents such as methylene dichloride, carbon tetrachloride, chloroform or dioxane are used, the product obtained contains large amounts of the undesired Δ² isomer.

The reaction is carried out at a temperature of from about −10° C. to about 45° C. and preferably at a temperature of from about 0° C. to about 25° C. The amount of N-methyl pyrrolidine is not critical, but preferably about 1 to about 2 equivalents of N-methyl pyrrolidine per equivalent of compound of formula IV.

The compound of the formula IV may be prepared by reaction of a solution of the compound of the formula V: 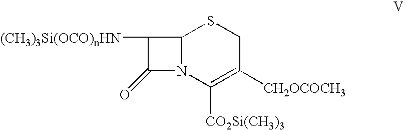
wherein n=0 or 1,
in a cyclohexane with trimethylsilyl iodide (TMSI). The compound of formula V, wherein n=0 is the preferred compound and is represented by formula Va:

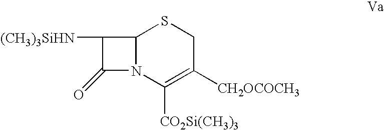

When cyclohexane is used as solvent, the compound of formula IV obtained is substantially free of the Δ2isomer. As it Is known from the description in U.S. Pat. No. 4,868,294, solvents such as 1,2-dichloroethane, chlorobenzene, dioxane and carbontetrachloride, yield compound IV containing significant amounts of the undesirable Δ² Isomer.

The reaction is carried out at a temperature of from about 0° C. to about 45° C. preferably at a temperature from about 5° C. to about 40° C. and more preferably at a temperature from about 5° C. to about 25° C. At least one equivalent of trimethylsilyl iodide is required to convert all the compound V to IV, preferable amount being about 0.9 to about 2.5 equivalents per equivalent of compound V, more preferable amount being about 1.0 to about 2.0 equivalents of trimethylsilyl iodide.

The compounds of formula Va may be prepared by reacting 7-amino cephalosporanic acid (7-ACA) of the formula VI: 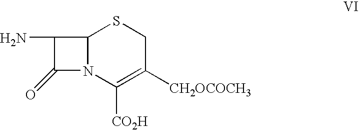
with hexamethyldisilazane (HMDS) at a temperature from about 0° C. to the boiling temperature of the cyclohexane. The reaction is preferably carried out in the presence of catalytic amount (about 0.05 to about 0.1 equivalent each per equivalent of 7-ACA) of imidazole and acetamide: or in the presence of catalytic amount (about 0.01 to about 0.1 equivalent per equivalent of 7-ACA) of trimethylsilyl iodide. The reaction is preferably carried out at a temperature from about 25° C. to the boiling temperature of cyclohexane, more preferably from about 35° C. to the boiling temperature of cyclohexane and most preferably at the boiling temperature of cyclohexane. It has been found that silylation occurs to a larger extent at a faster rate when the silylation is carried out at the boiling temperature of cyclohexane than when the silylation is carried out at a lower temperature. The HMDS may be used in an amount from about 0.9 to about 1.5 equivalents per equivalent of 7-ACA, preferably from about 1.0 to 1.4 equivalents of HMDS per equivalent of 7-ACA. The catalytic amounts of acetamide and imidazole may preferably used in the silylation step.

The compound of formula V, wherein n=1 may be prepared by bubbling carbon dioxide gas into a solution of compound Va in cyclohexane.

In an alternative preparation of compound of formula III, a solution of compound of formula V in cyclohexane is treated with N-methyl pyrrolidine followed by the addition of at least one equivalent of trimethylsilyl iodide. The reaction can be conducted at a temperature of from about 0° C. to about 45° C. and preferably from about 0° C. to about 25° C. The N-methyl pyrrolidine may be used in an amount d from about 1.0 to about 2.0 equivalents per equivalent of compound V. The trimethylsilyl iodide may be used in an amount of from about 0.9 to about 2.5 equivalents per equivalent of compound V, and preferably from about 1.0 to 1.8 equivalents.

In an another alternative preparation of compound III, a solution of compound V in cyclohexane is reacted with N-methyl-N-trimethylsilyl pyrrolidine iodide having the formula VII: 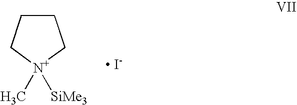
at a temperature from about 0° C. to about 45° C. and preferably from about 0° C. to about 25° C. The reaction may preferably be carried out in the presence of trimethylsilyl iodide in an amount from about 0.2 to about 0.8 equivalents per equivalent of compound V. The compound of formula VII may be used in an amount from about 1.0 to about 2.5 equivalents per equivalent of compound V and preferably from about 1.0 to about 2.0 equivalents of compound VII per equivalent of compound V.

The compound of formula VII may be prepared by reacting N-methyl pyrrolidine with about an equimolar amount of trimethylsilyl iodide in cyclohexane at a temperature of from about −10° C. to about 45° C. Preferably the reaction is carried out at a temperature of from about 0° C. to about 25° C., more preferably from about 0° C. to about 10° C.

In a preferred reaction scheme, the compound of formula II or the salt thereof is prepared from 7-ACA in a “one pot” reaction i.e., without the isolation of any intermediates using cyclohexane as main solvent thought out the reaction sequence.

The other compounds of formula I or their salts may be prepared by similar -procedure described for the compound II and its salts.

The “compound substantially free of Δ² isomer” refers to the compound containing the content of Δ² isomer in less than about 10% of the compound plus the isomer, preferably less than about 3% and more preferably less than about 0.4%.

The compounds of the formula I can be prepared by the sequence shown below in reaction scheme I.

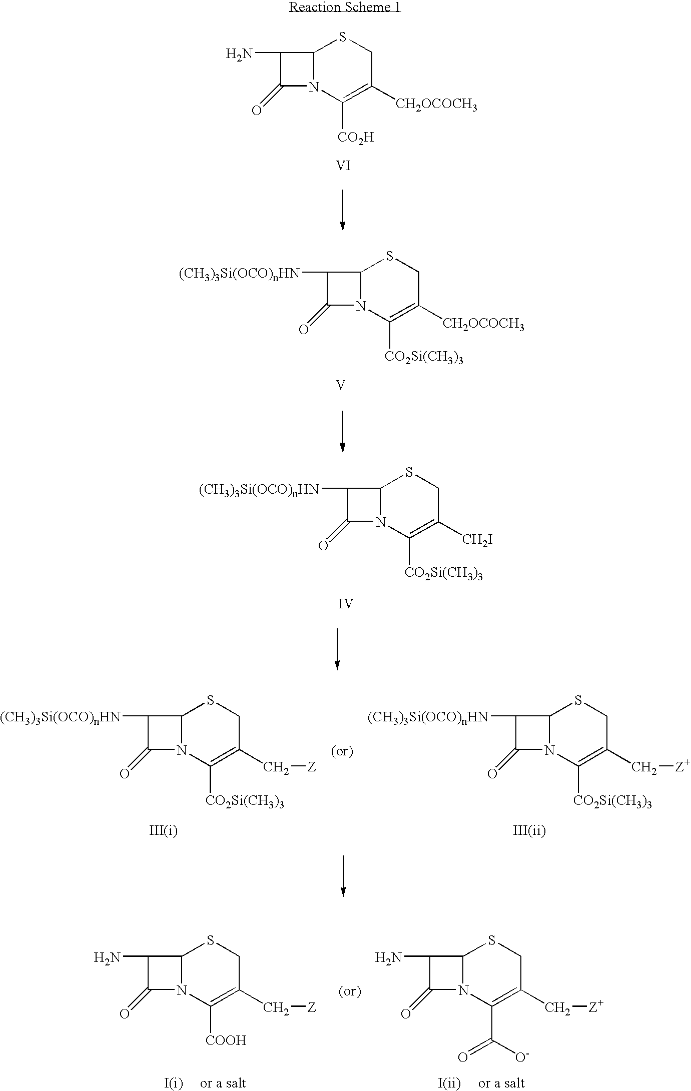

In the compounds of the formulas III(i), III(ii), IV and V, n=0 or 1 and in the compound of the formulas III(i) and III(ii), Z and Z⁺ have the same meaning as defined in formula in formulas I(i) and I(ii).

The compound of formula IV may be treated with appropriate HZ or Z⁻ to obtain to the compound of formula III(i) or with appropriate Z to obtain the compound of formula III(ii).

The compounds of formula I, II are readily converted to broad spectrum cephalosporin antibiotics by acylation with the appropriate side-chain acid. Some of the cephalosporin antibiotics that can be prepared include those described in U.S. Pat. No. 4,406,899, U.S. Pat. No. 4,168309, U.S. Pat. No. 4,223,135, U.S. Pat. No. 4,336,253, U.S. Pat. No. 4,379,787 and J. Organic Chemistry 1988, 53, 983-991. The acylation can be carried out by conventional means using for example acid chloride, mixed acid anhydrides and activated esters. For example the compound of formula II as HCl or Hl salt is converted to cefepime dihydrochloride monohydrate by N-acylating with syn-2-(2-aminothiazol4-yl)-2-methoxyimino acetyl chloride hydrochloride, syn-2-(2-aminothiazol-4-yl-2-methoxyimino acetic acid 2-benzothiazolyl thioester (MAEM) or syn-2-(2-aminothiazol-4-yl)-2-methoxyimino acetic acid 1-benzotriazolyl ester and then converting cefepime into cefepime dihydrochloride monohydrate using hydrochloric acid. The preferred method can be shown as in the scheme below: 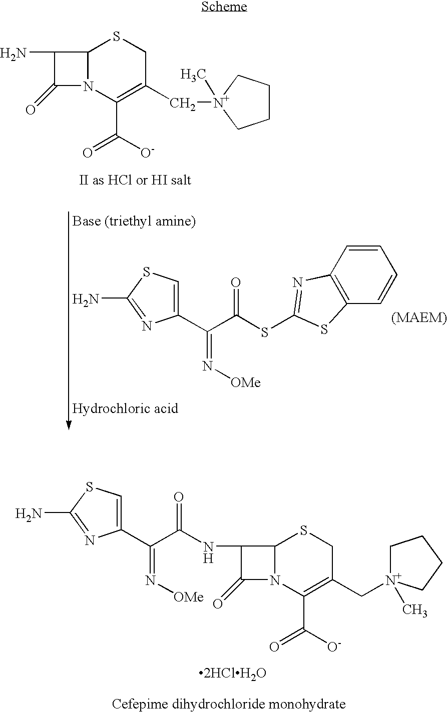
The invention will now be further described by the following examples, which are illustrative rather than limiting.

### EXAMPLE 1

7-Aminocephalosporanic acid (7-ACA) (200 gm) is stirred in cyclohexane (1400 ml) for 10 minutes at 25° C. and then acetamide (400 mg), imidazole (400 mg) and hexamethyldisilazane (142 gm) are added to the reaction mass at 25° C. The reaction mass is slowly heated to reflux temperature and stirred for 2 hours at the reflux to form a clear solution. The reaction mass is distilled to collect about 100 ml cyclohexane and then the mass is cooled to 5° C. to give the reaction mass containing (6R,7R)-3-[(Acetyloxy)methyl]-7-trimethylsilyl) aminoceph-3-em-4-oic acid.

Trimethylsilyl iodide (246 gm) is slowly added to the mixture of N-methylpyrrolidine (94 gm) and cyclohexane (700 ml) at 5-10° C. over a period of 30 minutes. Then reaction mass is stirred for 30 minutes at 5-10° C. To this mass is added to the reaction mass containing (6R, 7R)-3-[(acetyloxy)methyl]-7-(trimethylsilyl)aminoceph-3-em-4-oic acid over a period of 30 minutes at 5-10° C. and then trimethylsilyl iodide solution (66 gm in 75 ml cyclohexane) is added at 5-10° C. in 15 minutes. The mass is heated to 37-40° C. in 30 minutes and stirred for 35 hours at the same temperature.

The reaction mass is then cooled to 5° C., isopropyl alcohol (100 ml) is added at 5-10° C. Concentrated HCl (200 ml) and water (400 ml) are slowly added over a period of 20 minutes at 5-10° C. The reaction mass is stirred for 15 minutes. The layers are separated and organic. layer is extracted with water (100 ml). Then the combined aqueous layer is cooled to 5-10° C., subjected to carbon treatment and filtered on hyflo-bed. The filtrate is cooled to 5° C. Isopropyl alcohol (4000 ml) is added to the filtrate over a period of one hour at 5-10° C. Then the solid precipitated is filtered, washed with isopropyl alcohol (100 ml) and then dried at 40-45° C. under vacuum to give 172 gm of [6R-(6α,7β)]-1-[[7-Amino-2-carboxy-8-oxo-5-thia-1-azabicyclo[4.2.0]oct-2-en-3-yl]methyl]-1-methylpyrrolidinium inner salt hydrochloride (HPLC purity 98.77%, 0.08% Δ²isomer).

### EXAMPLE 2

[6R-(6α,7βˆ-i-tˆ-Aminoˆ-carboxy-δ-oxo-S-thia-i-azabicyclo [4.2.0]oct-2-en-S-yl]methyl]-1-methylpyrrolidinium inner salt hydrochloride (25 gm obtained as in example I) is added to a mixture of water (200 ml) and acetone (375 ml) at 5° C. and stirred for 10 minutes and syn-2-(2-aminothiazol-4-yl)-2-methoxyimino acetic acid 2-benzothiazolyl thioester (MAEM) (34.10 gm) is added at 5-10° C. Triethylamine is slowly added to the reaction mixture at 5-10° C. to adjust the pH to 7.5-7.7 and stirred for 10 minutes at 5-10° C. The temperature of the reaction mass is then slowly raised to 20- 25° C. and maintained for 4 hours 30 minutes. Ethyl acetate (250 ml) is added to the reaction mass at 5° C., stirred for 15 minutes and the layers are separated. Then the aqueous layer is extracted with ethyl acetate (125 ml) at 5-10° C. The aqueous layer is subjected to carbon treatment and filtered on hyflo-bed. 10 N HCl (60 ml) and acetone (400 ml) are added to the filtrate at 5-10° C., seeded with cefepime dihydrochloride monohydrate (0.5 gm) and stirred for 30 minutes at 5-10° C. Acetone (850 ml) is added the filtrate for 30 minutes at 5-10° C., cooled to 0-5° C. and maintained for 1 hour. Then the separated solid is filtered, washed with acetone (150 ml) and dried to give 32.8 gm of 7-[α-(2-aminothiazol-4-yl)-α-(z)-methoxyimino acetamido]-3-[(1-methyl-1-pyrrolidinio)methyl]-3-cephem-4-carboxylate dihydrochloride monohydrate (cefepime dihydrochloride monohydrate) (HPLC purity 99.92%, 0.06% Δ² isomer).

### EXAMPLE 3

Stage-I:

(6R, 7R)-Trimethylsilyl 7-(trimethylsilyl)amino-3-acetoxymethylceph-3-em-4-carboxylate:

7-Amino cephalosporanic acid (30 gm) is suspended in cyclohexane (210 ml) at 25° C., then hexamethyidisilazane (27.84 ml), acetamide (60 mg) and imidazole (60 mg) are added at 25° C. and the reaction mass is heated to reflux for 3 hours. Then the solution obtained is cooled to 25° C. to give the title compound in cyclohexane.

Stage H:

(6R, 7R)-Trimethylsilyl 7-(trdmethylsilyl)amino-3-iodomethylceph-3-em-4-carboxylate:

The solution of (6R, 7R)-Trimethylsilyl 7-(trdmethylsilyl)amino-3-acetoxy methylceph-3-em4-carboxylate in cyclohexane obtained in stage-I is cooled to 0-5° C., the solution of trimethylsilyl iodide (48 gm) in cyclohexane (55 ml) is slowly added over a period of 30 minutes and stirred for 1 hour at 0-5° C. to give the title compound solution in cyclohexane.

Stage-III:

(6R, 7R)-Trimethylsilyl 7-(trimethylsilyl)amino-3-(1-methyl-1-pyrrolidinio)methyl ceph-3-em-4-carboxylate iodide:

The solution of (6R, 7R)-Trimethylsilyl 7-(trimethylsilyl)amino-3-iodomethylceph-3-em-4-carboxylate in cyclohexane obtained in stage-II is added to a solution of N-methyl pyrrolidine (17.3 ml) in cyclohexane (50 ml) and stirred for 30 minutes at 0-5° C. Then the temperature of the reaction mass is raised to 38-40° C. and stirred for 30 hours to give the title compound solution in cyclohexane.

Stage-IV:

(6R,7R)-7-amino-3-(1-methyl-1-pyrrolidinioJmethylceph-S-mˆ-carboxylic acid hydrochloride:

The solution of (6R, 7R)-Trimethylsilyl 7-(trimethylsilyl)amino-3-(1-methyl-1-pyrrolidinio)methylceph-3-em-4-carboxylate iodide in cyclohexane as obtained in stage-III is cooled to 0-5° C. and isopropyl alcohol (15 ml) is slowly added. Then the mixture of concentrated HCl (30 ml) and water (60 ml) is added to the reaction mass at 8-10° C. and the layers are separated. The aqueous layer is subjected to carbon treatment, filtered and cooled to 8-10° C. Then isopropyl alcohol (600 ml) is added slowly to the aqueous layer and the title compound is precipitated. The precipitated solid is filtered, washed with isopropyl alcohol (20 ml) and dried under vacuum at 40° C. for 8 hours to 10 hours to give 16 gm of (6R, 7R)-7-amino-3-(1-methyl-1-pyrrolidinio)methylceph-3-em-4-carboxylic acid hydrochloride (0.06% Δ² isomer).

Stage-V:

Methoxyimino-[2-amino-4-thiazolyl]acetyl chloride hydrochloride (10.9 gm) and (6R,7R)-7-amino-3-1-methyl-1-pyrrolidinio)methylceph-3-em-4-carboxylic acid hydrochloride (15 gm) are added to a mixture of water (100 ml) and acetone (150 ml) and cooled to 8-10° C. The pH of the reaction mass is adjusted to 7.2-7.5 with triethylamine and then stirred for 4 hours at 10° C. Ethyl acetate (150 ml) is added to the reaction mass, stirred for 30 minutes and separated the layers. The aqueous layer is then subjected to carbon treatment, stirred for 30 minutes and filtered. The mixture of acetone (36 ml) and concentrated HCl (36 ml) is added to the filtrate at 5° C. Acetone (700 ml) is added and cooled to 0- 5° C. Then the separated solid is filtered, washed with acetone (50 ml) and dried under vacuum at 40° C. for 10 hours to give 18 gm of 7-[α-(2-aminothiazo[-4-yl)-α-(Z)-methoxyiminoacetamido]-3-[(1-methyl-1-pyrrolidinio)methyl]-3-cephem-4-carboxylate dihydrochloride monohydrate (cefepime dihydrochloride mono hydrate) (HPLC purity 99.82%, 0.05% Δ² Isomer).

1. A process for the preparation of the compound of formula II: 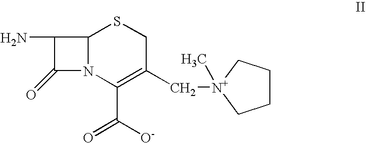
or a salt thereof which is substantially free of the Δ² isomer, which comprises treating the compound of formula III: 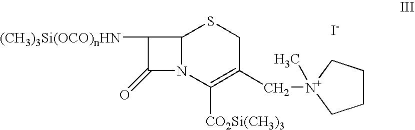
wherein n=0 or 1,
in cyclohexane with a C₁- C₄-alkanol or water to remove silyl protecting groups, optionally converting to the salt of the compound of formula II.

2. The process according to claim 1, wherein the salt is hydrochloride or hydroiodide salt.

3. The process according to claim 1, wherein the compound of the formula III used is the compound IIIa;

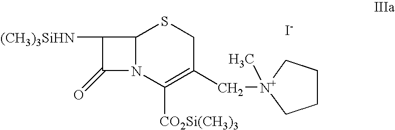

4. The process according to claim 1, wherein the C₁-C₄-alkanol is selected from the group consisting of isopropyl alcohol, methanol and ethanol.

5. The process according to claim 4, wherein the C₁-C₄-alkanol is isopropyl alcohol.

6. A process for the preparation of the compound of formula II: 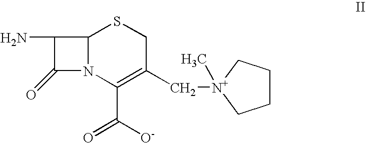
or a salt thereof which is substantially free of the Δ² isomer, comprising the steps of:
a) reacting the compound of formula IV: 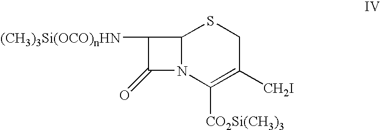
wherein n=0 or 1,
in cyclohexane with N-methylpyrrolidine to produce the compound of formula III: 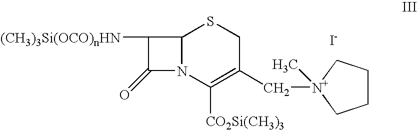
wherein n=0 or 1, and
(b) treating the compound of formula III in cyclohexane with a C₁-C₄-alkanol or water to remove silyl protecting groups, optionally converting to the salt of the compound of formula II.

7. The process according to claim 6, wherein the conversion into the salt in step (b) is carried out by treating the compound of formula II with hydrochloric acid or hydroiodic acid.

8. The process according to claim 6, wherein the compound of formula IV used is the compound IVa: 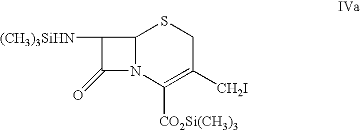
to obtain the compound formula IIIa:

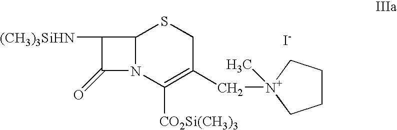

9. A process for the preparation of the compound of formula II: 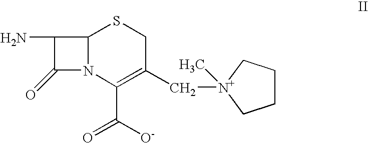
or a salt thereof which is substantially free of the Δ² isomer, comprising the steps of:
a) reacting the compound of formula V: 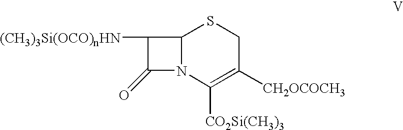
wherein n=0 or 1,
in cyclohexane with at least one equivalent of trimethylsilyl iodide per equivalent of compound of formula V to produce the compound of formula IV:
wherein n=0 or 1, 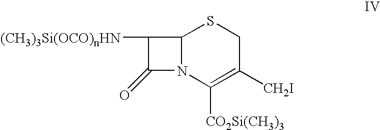
b) reacting the compound of formula IV in cyclohexane with N-methylpyrrolidine to produce the compound of formula III: 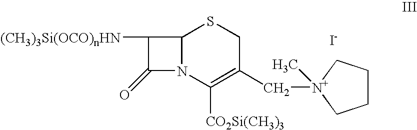
wherein n=0 or 1, and
(c) treating the compound of formula III in cyclohexane with a C₁-C₄-alkanol or water to remove silyl protecting groups, optionally converting to the salt of the compound of formula II.

10. The process according to claim 9, wherein the salt is hydrochloride or hydroiodide salt.

11. The process according to claim 9, wherein the compound of the formula V used is the compound Va;

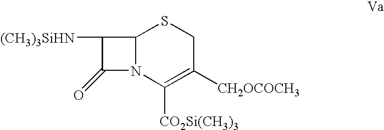

12. A process for the preparation of the compound of formula I(i) or I(ii): 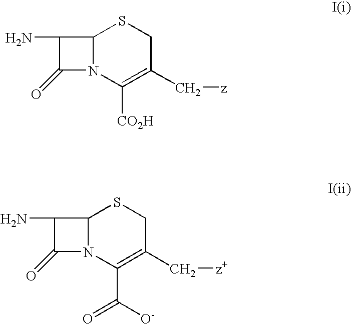
or a salt thereof which is substantially free of the Δ² isomer, comprising the steps of:
a) treating the compound of formula VI: 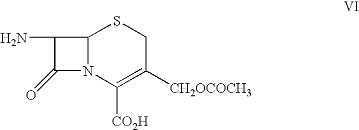
in cyclohexane with at least one equivalent of hexamethyldisilazane per equivalent of compound of formula VI and catalytic amount of trimethylsilyl iodide to produce the compound of formula V: 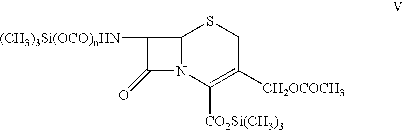
wherein n=0 or 1,
b) treating the compound of formula V in cyclohexane with at least one equivalent of trimethylsilyl iodide per equivalent of compound of formula V to produce the compound of formula IV: 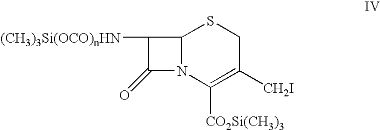
wherein n=0 or 1,
c) reacting the compound of formula IV in cyclohexane with Z⁻ or HZ to produce the compound of formula 111(i) or with Z to produce the compound of formula IIII(ii): 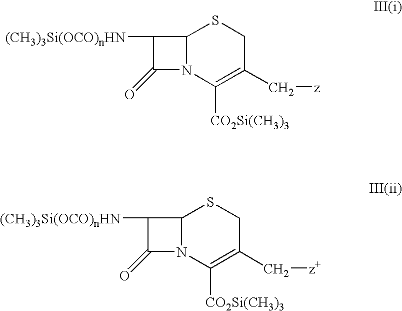
wherein n=0 or 1,
Z is 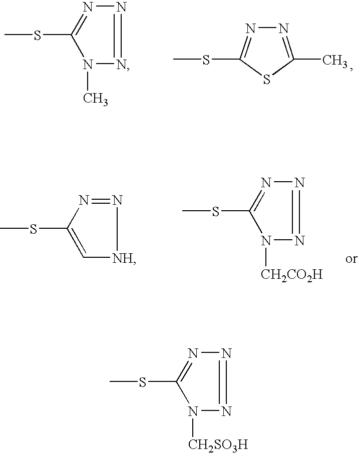
and
z⁺ is 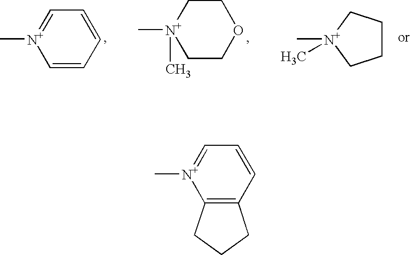
(d) treating the compound of formula III(i) or III(ii) in cyclohexane with a C₁-C₄-alkanol or water to remove silyl protecting groups, optionally converting to the salt of the compound of formula II.

13. The process according to claim 12, wherein the compound produced in step (c) is III(i) wherein Z⁺ 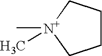
and n=0.

14. The process according to claim 12, wherein the salt is hydrochloride or hydroiodide salt.

15. A process for the preparation of the compound of formula II: 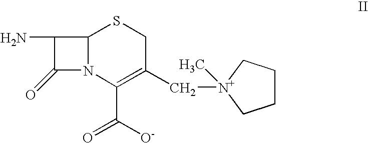
or a salt thereof which is substantially free of the Δ² isomer, which comprises treating a solution of the compound of formula V: 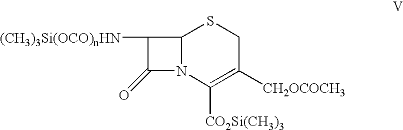
wherein n=0 or 1,
in cyclohexane with at least one equivalent of N-methylpyrrolidine then with at least one equivalent of trimethylsilyl iodide per equivalent of compound of formula V, followed by treatment with a C₁-C₄-alkanol or water to remove silyl protecting groups, optionally converting to the salt of the compound of formula II.

16. The process according to claim 15, wherein the salt is hydrochloride or hydroiodide salt.

17. The process according to claim 15, wherein the compound of the formula V used is the compound Va;

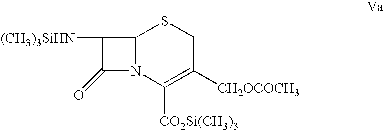

18. A process for the preparation of the compound of formula II: 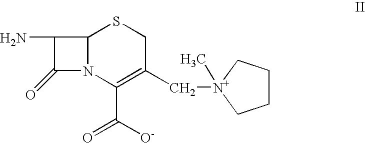
or a salt thereof which is substantially free of the Δ²isomer, which comprises treating a solution of the compound of formula V: 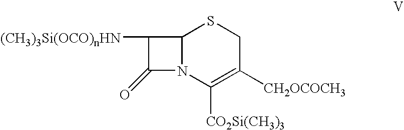
wherein n=0 or 1,
in cyclohexane with the compound of formula VII: 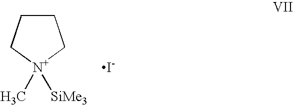
in cyclohexane, followed by treatment with a C₁- C₄-alkanol or water to remove silyl protecting groups, optionally converting to the salt of the compound of formula II.

19. The process according to claim 18, wherein the salt is hydrochloride or hydroiodide salt.

20. The process according to claim 18, wherein the compound of the formula V used is the compound Va;

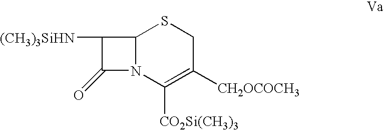

21. A process for the preparation of the compound of formula II: 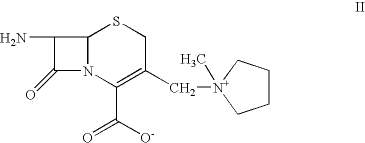
or a salt thereof which is substantially free of the Δ² isomer, which comprises treating a solution of the compound of formula VI: 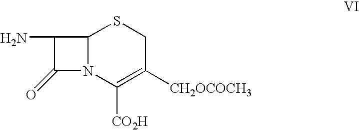
in cyclohexane with at least one equivalent of hexamethyldisilazane per equivalent of compound VI and then with the compound of formula VII: 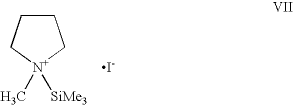
in cyclohexane, followed by treatment with a C₁- C₄-alkanol or water to remove silyl protecting groups, optionally converting to the salt of the compound of formula II.

22. The process according to claim 21, wherein the salt is hydrochloride or hydroiodide salt.

23. The process according to claim 21, wherein the reaction with the compound VII is carried out in the presence of trimethylsilyl iodide.

24. The process according to claim 21, wherein the reaction with hexamethyldisilazane is carried out in the presence of the catalytic amounts of imidazole and acetamide.

25. The process according to claim 21, wherein the reaction with hexamethyidisilazane is carried out in the presence of the catalytic amount of trimethylsilyliodide.

26. The process according to claim 3, wherein the C₁- C₄-alkanol is selected from the group consisting of isopropyl alcohol, methanol and ethanol.

27. The process according to claim 26, wherein the C₁-C₄-alkanol is selected from the group consisting of isopropyl alcohol, methanol and ethanol.

28. The process according to claim 7, wherein the compound of formula IV used is the compound IVa: 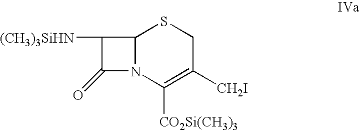
to obtain the compound formula IIa: [chemical structure]
